(19) United States
(12) Patent Application Publication
(10) Pub. No.: US 2016/0270969 A1
(43) Pub. Date: Sep. 22, 2016

(54) Fluid Absorber

(71) Applicant: Shane L. Hudson, Reno, NV (US)

(72) Inventor: Shane L. Hudson, Reno, NV (US)

(21) Appl. No.: 15/074,088

(22) Filed: Mar. 18, 2016

Related U.S. Application Data
(60) Provisional application No. 62/136,181, filed on Mar. 20, 2015.
(60) Provisional application No. 62/215,694, filed on Sep. 8, 2015.
(60) Provisional application No. 62/237,968, filed on Oct. 6, 2015.
(60) Provisional application No. 62/238,599, filed on Oct. 7, 2015.

(51) Int. Cl. A61F 13/20; A61F 13/26

(52) U.S. Cl. CPC A61F 13/2002; A61F 13/266

(57) ABSTRACT

A fluid absorber. The absorber includes a handle and an absorbent material attached to a distal extremity of the handle. The absorbent material has a non-circular cross section. An optional housing encloses the absorbent material until the absorbent material is ejected from the housing for use.

### CROSS-REFERENCE TO RELATED APPLICATIONS

This application claims priority from the following U.S. Provisional patent applications:

Ser. No. 62/136,181 filed 20 Mar. 2015,

Ser. No. 62/215,694 filed 8 Sep. 2015,

Ser. No. 62/237,968 filed 6 Oct. 2015, and

Ser. No. 62/238,599 filed 7 Oct. 2015,

the entire contents of which are incorporated herein.

### BACKGROUND

Women have long confronted the problem of retention and discharge of semen from the vaginal area after engaging in intercourse. One common way of removing this fluid is by douching, which involves components such as a squeeze bottle, a douche bag, and additional flushing liquid to wash out the vaginal area and any fluid that is in it. Some women have used tampons to remove fluid from the vaginal area after engaging in intercourse. In another approach, absorbent cloth has been used to physically wipe the vaginal area.

### SUMMARY

The applicant believes he has discovered some of the problems with devices and methods that women have used to remove semen from their vaginal areas. Douching is inconvenient and can be messy, especially if a bidet is not available. Some health-care professionals believe that douching can actually be dangerous in that it can interfere with normal self-cleaning of the vagina and with the natural bacterial culture of the vagina. Tampons do not readily absorb semen and can present other problems of their own. Using absorbent cloth can be slow and, as with other methods, can cause vaginal irritation and infection.

Briefly and in general terms, an embodiment of a fluid absorber for absorbing semen after intercourse includes a rigid handle and an absorbent material attached to a distal extremity of the handle. The absorbent material may have a non-circular cross-section for comfortable insertion into the vagina. The absorbent material may have an ellipsoidal, square or rectangular, pentagonal, hexagonal, octagonal, or other cross-sectional shape. Some of these shapes may present flat as well as curved areas that extend longitudinally along the absorbent material. The absorbent material may be tapered toward the distal extremity of the handle for easier insertion.

In some versions the absorbent material comprises cotton. The cotton may be sterilized and enclosed in a wrapper, for example a wrapper made of paper, to keep it clean. The wrapper may be removed just before using the fluid absorber; the wrapper may be kept and used to wrap the used fluid absorber for convenient disposal. The cotton may be of relatively light density so as to readily absorb viscous fluids such as semen.

In some embodiments the handle may comprise rolled paper, cardboard, plastic, or the like. The handle is relatively rigid compared with the absorbent material but may be flexible. The absorbent material may be attached to the handle with non-toxic glue or the like.

Another embodiment has a tubular housing, for example of paper, cardboard, or plastic, enclosing the absorbent material. The handle may extend through an opening in a proximal extremity of the housing. A distal extremity of the housing may be petaled so that the fluid absorber may conveniently be ejected from the housing simply by pushing on the handle while grasping the housing, avoiding any contact with the absorbent material prior to insertion.

Another embodiment has a grip, for example a relatively rigid disk-shaped material, affixed to a proximal end of the housing. The handle extends through an opening in the grip. The fluid absorber may be ejected from the housing by pressing the handle while holding a finger under the grip on a side opposite the handle.

A method of using a fluid absorber includes removing any protective wrapper; grasping the handle; ejecting the fluid absorber from any housing, for example by pressing on the handle while holding a finger under a grip affixed to the housing or while grasping the housing; inserting the absorber; leaving it in place long enough for the absorber to absorb semen, again grasping the handle; pulling the fluid absorber out; and discarding it. In an embodiment that includes a housing, the housing may be ejected prior to insertion into the vagina, or the absorber including the housing may be inserted and then the handle pressed to eject the absorbent material from the housing and push the absorbent material deeper into the vagina.

The scope of the inventive features disclosed in this Specification is not limited to the features and issues described above. There are other novel aspects, problem solutions, and advantages that will become apparent from the detailed description and drawings, illustrating by example the principles of the invention.

### BRIEF DESCRIPTION OF THE DRAWINGS

FIG. 1 is a perspective view of an embodiment of a fluid absorber.

FIGS. 2A and 2B provide a side view and a cross-section view of another embodiment of a fluid absorber.

FIG. 3 is a side view of an embodiment similar to that shown in FIGS. 2A and 2B but with tapered absorbent material.

FIGS. 4A through 4H are cross-sectional views of embodiments similar to the embodiment shown in FIGS. 2A and 2B but with different cross-sectional shapes.

FIGS. 5A and 5B provide side and cross-sectional views of another embodiment of a fluid absorber.

FIGS. 6A and 6B are end views of embodiments similar to the embodiment shown in FIGS. 5A and 5B but with differing arrangements of slits that form the petaling.

FIG. 7 is a side view of another embodiment of a fluid absorber.

FIG. 8 is a side view of another embodiment of a fluid absorber.

FIG. 9 is a flow chart of a method of using a fluid absorber.

FIG. 10 is a flow chart of another method of using a fluid absorber.

### DETAILED DESCRIPTION

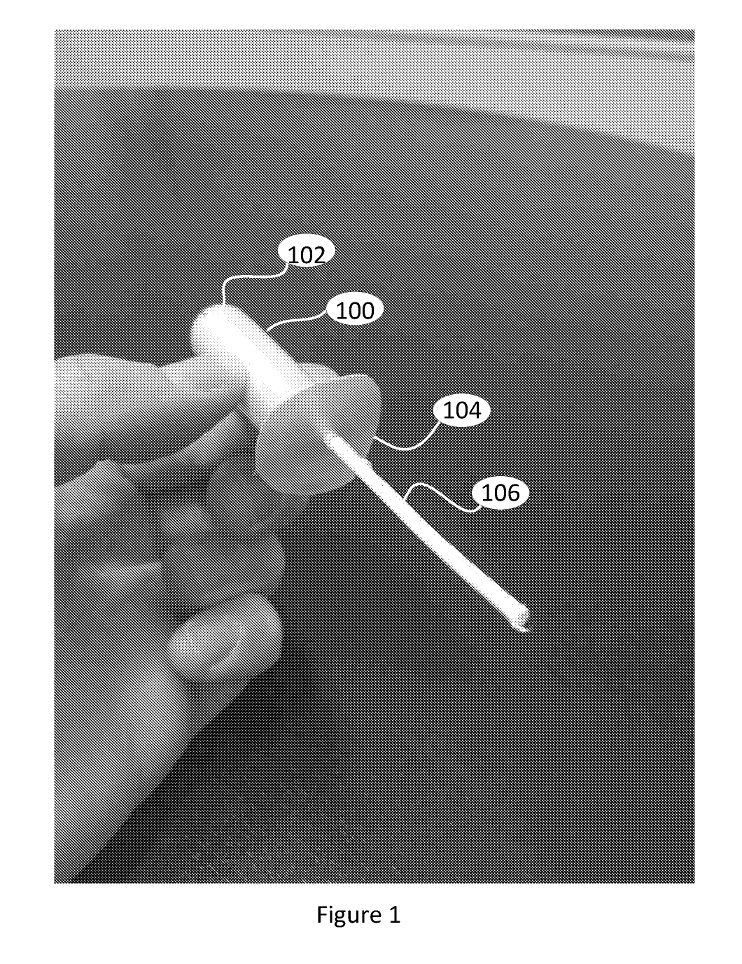

FIG. 1 gives a perspective view of an embodiment of a fluid absorber being held in a hand. Absorbent material (concealed from view) is enclosed in a housing 100 that may be made of paper, cardboard, flexible plastic, or other suitable material. A distal extremity 102 of the housing is petaled to facilitate ejection of the fluid absorber. A gripper 104 in the shape of a disk is affixed to a proximal extremity of the housing. A handle 106 protrudes through an opening in the gripper.

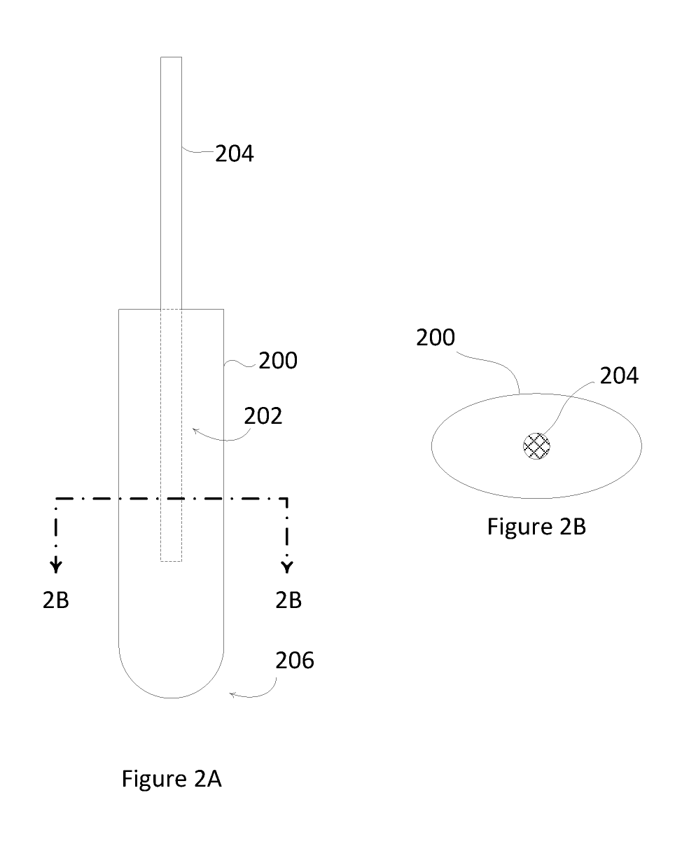

Another embodiment of a fluid absorber is shown in FIGS. 2A and 2B. An absorbent material 200 is attached to a distal extremity 202 of a rigid handle 204. The absorbent material 200 has a non-circular cross-section. For example, the absorbent material may have an ellipsoidal cross-section as best shown in FIG. 2B.

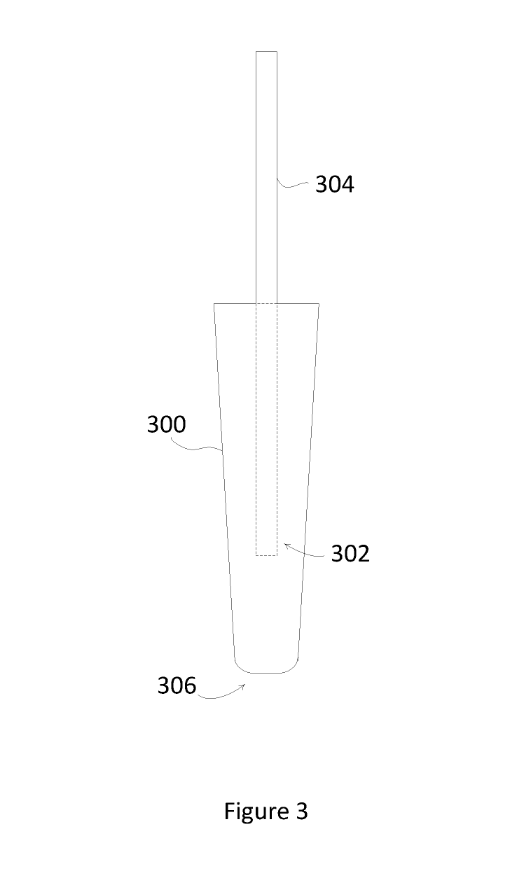

In another embodiment as shown in FIG. 3, a fluid absorber includes absorbent material 300 that is tapered to a narrower cross-section toward a distal extremity 302 of a handle 304.

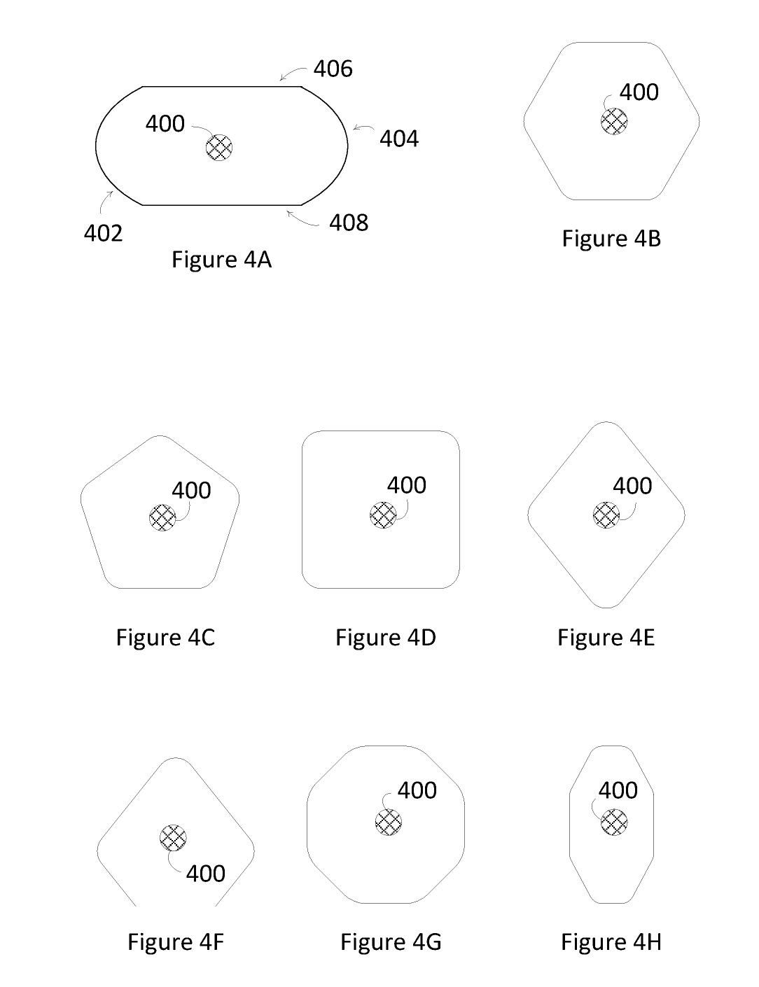

The absorbent material may have other cross-sectional shapes for comfort and convenience. For example, the absorbent material may be shaped as a flattened ellipsoid (FIG. 4A), a hexagon (FIG. 4B), a pentagon (FIG. 4C), a square (FIG. 4D) or other rectangle, a parallelogram such as a diamond shape (FIG. 4E), a flattened diamond (FIG. 4F), an octagon (FIG. 4G), or an attenuated octagon (FIG. 4H). In each figure the handle is indicated by the numeral 400.

Several of the shapes shown in FIGS. 4A through 4H provide one or more flat surfaces as well as curved surfaces that extend longitudinally along the absorbent material. For example, the flattened ellipsoid of FIG. 4A has curved surfaces 402 and 404 and flat surfaces 406 and 408 that extend longitudinally along all or a portion of the absorbent material for comfortable insertion and removal.

In the embodiment shown in FIG. 2A, the absorbent material 200 has a rounded extremity 206. This is not required and in some embodiments the absorbent material may be squared off or shaped otherwise. For example, in the embodiment shown in FIG. 3 the absorbent material 300 has a partially squared-off distal extremity 306.

The absorbent material in the various embodiments is selected for comfort during insertion and removal, and for its ability to absorb a viscous fluid such as semen. For example, cotton having a density about the same as that of an ordinary cotton ball may be used. Retaining such material in the vagina for about two minutes facilitates absorption and removal of all or most of the semen that may be present after intercourse.

The handle in the various embodiments is made of material that is relatively rigid compared with the absorbent material to facilitate easy insertion and removal of the fluid absorber. Examples of suitable material for the handle include paper, rolled paper, cardboard, wood similar to that used for sticks in Popsicles, and plastic.

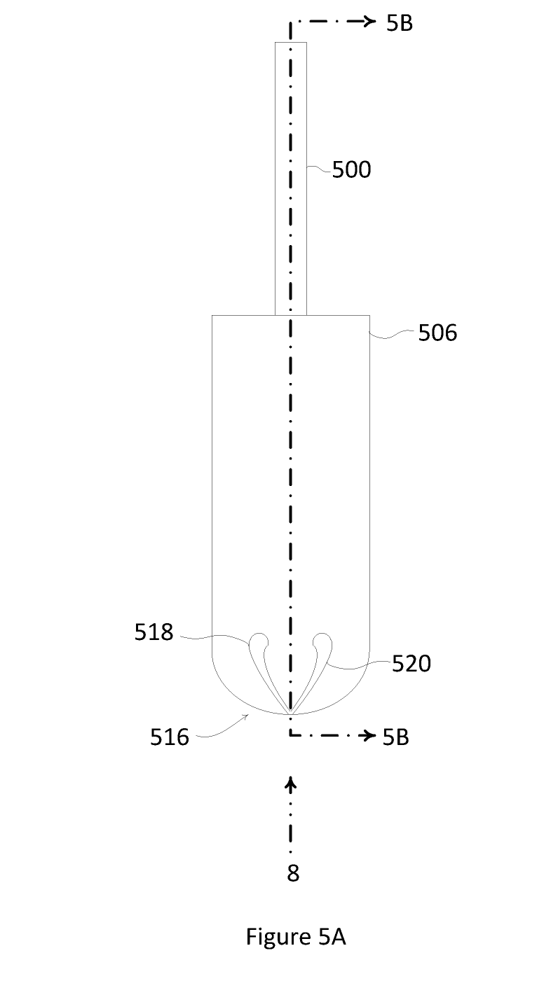

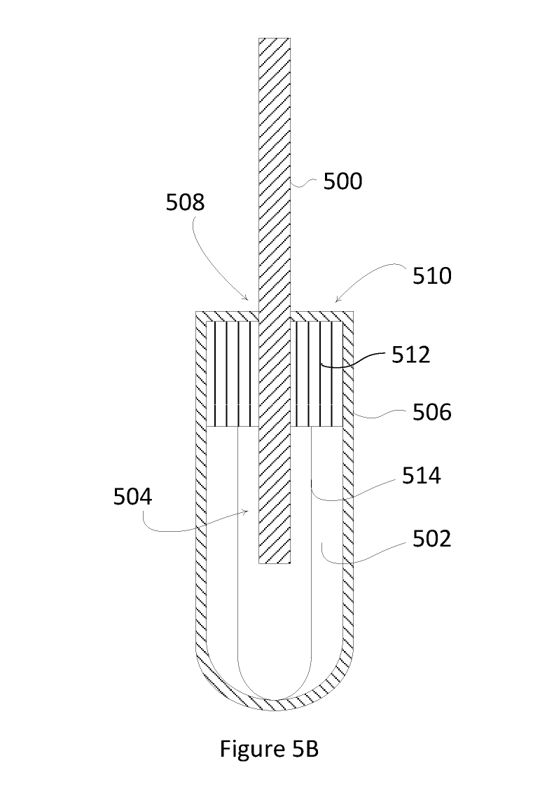

Another embodiment of a fluid absorber is shown in FIGS. 5A and 5B. This embodiment includes a rigid handle 500, an absorbent material 502 attached to a distal extremity 504 of the handle, and a housing 506 enclosing the absorbent material. The handle 500 extends through an opening 508 in a proximal extremity 510 of the housing. The absorbent material 502 may not fill the entire housing 506, in which case the housing encloses an empty space 512 between the absorbent material 502 and the distal extremity 510 of the housing.

The absorbent material has a non-circular cross section as described above with reference to other embodiments. In some embodiments the cross-section has one or more flat portions. As an example, in the embodiment of FIG. 5B the absorbent material has a flat portion 514.

The housing may comprise paper, cardboard, plastic, or other material.

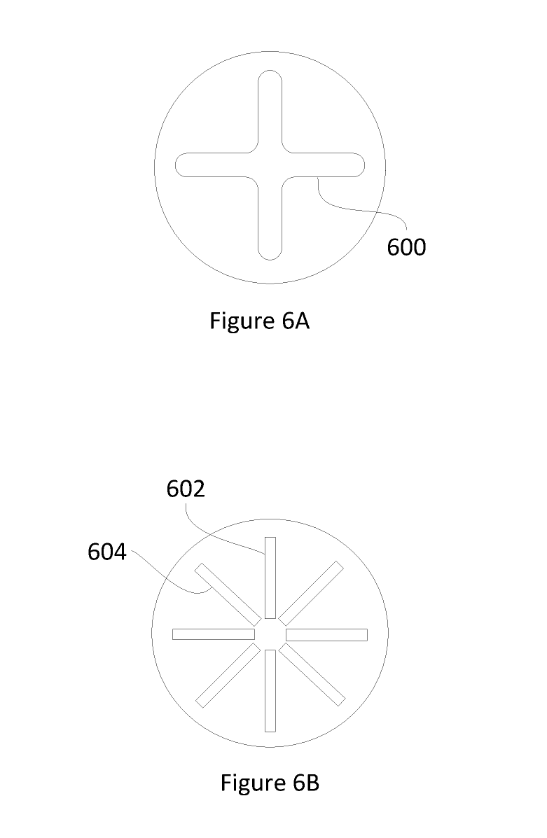

A distal extremity 516 of the housing may be petaled with slits such as a slit 518 and a slit 520 to facilitate ejecting the absorbent material (and in some embodiments the handle as well) out of the housing through the distal extremity of the housing. As shown in FIGS. 6A and 6B, petaling may take the form of a single cut such as a multi-fingered cut 600 in FIG. 6A or a plurality of individual slits such as slits 602 and 604 in FIG. 6B. Cuts or slits may be formed in a paper or other housing such that they are closed to protect the absorbent material from contamination until it is ejected from the housing. For example, pressure of a finger on the handle pushes the absorbent material out through the housing which separates and opens along the slits to permit easy ejection of the fluid absorber. In some embodiments the absorbent material may be ejected from the housing and then inserted into the vagina or ejected and inserted in one continuous motion, but in other embodiments the entire fluid absorber including the housing is inserted into the vagina, then the absorbent material is ejected from the housing by pressure on the handle and is inserted deeper into the vagina.

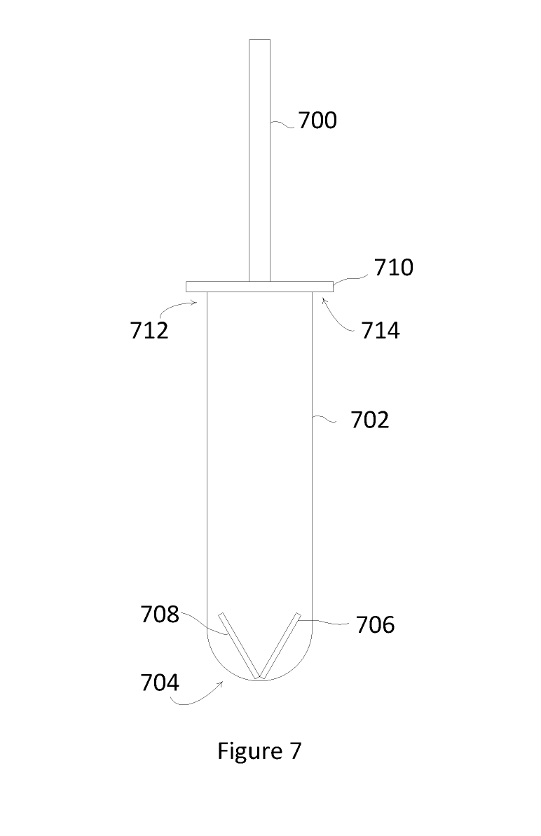

FIG. 7 illustrates another embodiment of a fluid absorber. This embodiment includes a handle 700, a housing 702, and absorbent material (concealed by the housing) attached to a distal extremity of the handle 700. A distal extremity 704 of the housing 702 is petaled with slits 706 and 708 to facilitate ejection of the absorbent material and handle from the housing. A gripper 710 is affixed to a proximal end 712 of the housing. The handle 700 extends through an opening in the gripper. The gripper 710 may be formed in a disk shape similar to the gripper 104 of FIG. 1. The gripper may be affixed to the housing with non-toxic glue or in some other way. The gripper may comprise paper, cardboard, plastic, or other suitable material. The gripper may be formed integrally with the housing from a single piece of material or may be formed separately and then attached to the housing.

In operation, the gripper 710 may be held with one hand while the handle 700 is pushed with the other hand to eject the absorbent material from the housing 702. Or a finger or thumb may be placed under a lip of the gripper adjacent the housing at a location 714 while another finger or thumb of the same hand presses on the handle to eject the absorbent material and handle from the housing.

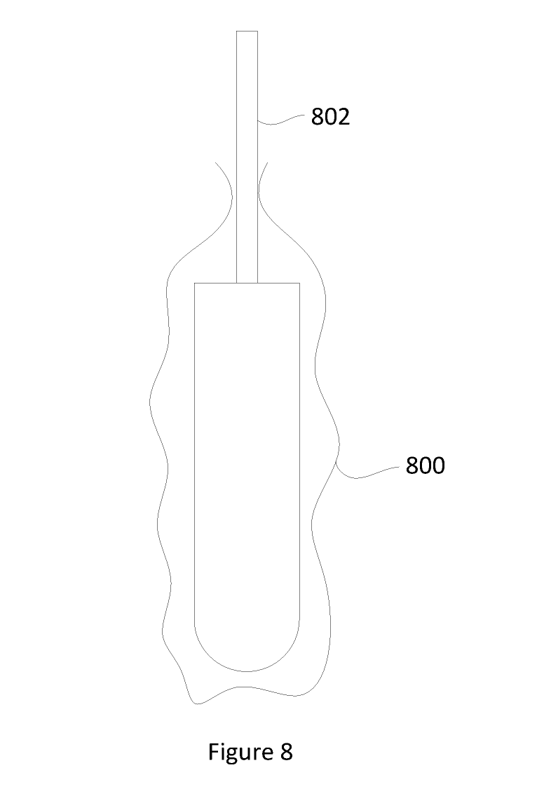

Some embodiments include an outer wrapper 800 enclosing a fluid absorber 802 as shown in FIG. 8. The wrapper may be made of paper, plastic, or other suitable protective material. The wrapper is removed before insertion of the absorbent material. The wrapper may be retained for wrapping the absorbent material after use for convenient disposal.

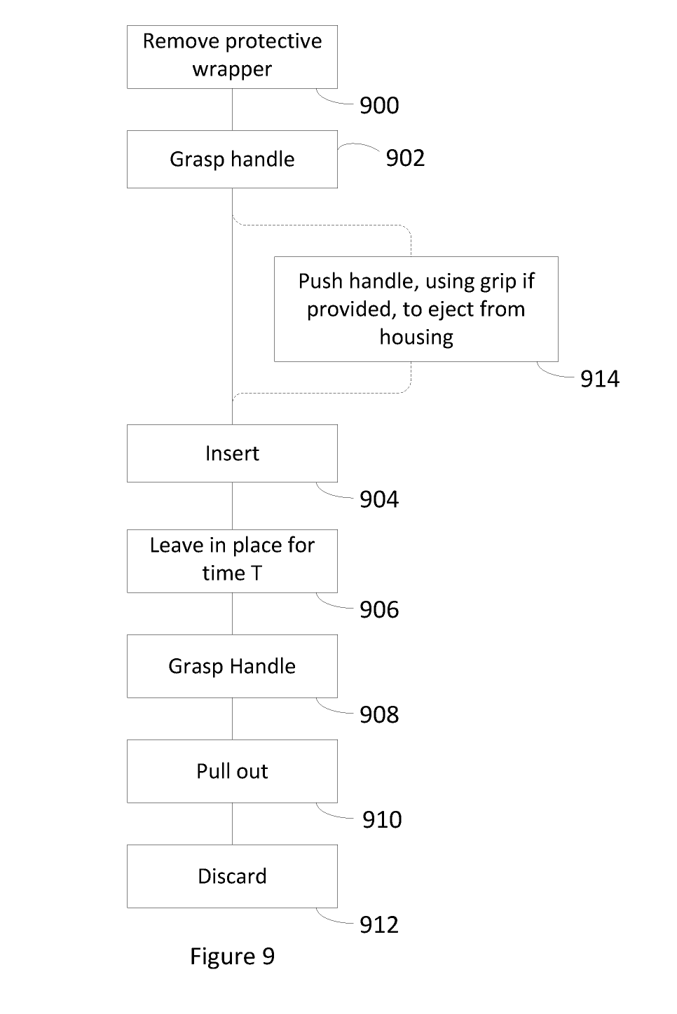

FIG. 9 illustrates a method of removing fluid from a vagina after intercourse. The method includes: (900) removing any protective wrapper from a fluid absorber of the kind having absorbent material carried by a handle; (902) grasping the handle; (904) inserting the absorbent material into the vagina; (906) leaving the absorbent material in place for an interval to time T sufficient to absorb fluid in the vagina; (908) grasping the handle; (910) pulling the absorbent material out of the vagina; and (912) discarding the fluid absorber.

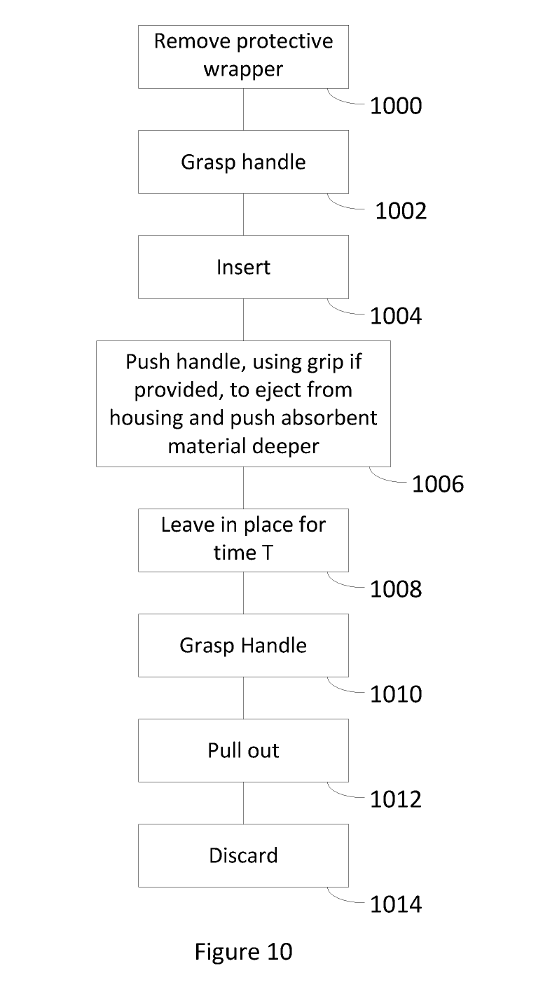

FIG. 10 illustrates another method of removing fluid from a vagina after intercourse. The method includes: (1000) removing any protective wrapper from a fluid absorber of the kind having absorbent material carried by a handle and enclosed in a housing; (1002) grasping the handle; (1004) inserting the fluid absorber including the housing into the vagina; (1006) pushing on the handle, using a grip if provided, to eject the absorbent material from the housing and push the absorbent material deeper into the vagina; (1008) leaving the absorbent material in place for an interval to time T sufficient to absorb fluid in the vagina; (1010) grasping the handle; (1012) pulling the fluid absorber out of the vagina; and (1014 discarding the fluid absorber.

It may be convenient to use two hands during insertion, one hand to hold the handle while the other holds the grip or a proximal end of the housing, so that the handle can be pushed in while the housing is held fixed after the fluid absorber has been inserted into the vagina, so that the absorbent material is ejected from the housing and pushed deeper into the vagina. Or one hand may be used, using one finger or the thumb to hold the grip while another finger or the thumb presses on the handle.

In some embodiments the time for absorbing fluids is about two minutes. In other embodiments the time may be as long as about ten minutes.

The invention is not to be limited by any of the foregoing or by the drawings, but only by the claims.

I claim:

1. A fluid absorber comprising a rigid handle and an absorbent material attached to a distal extremity of the handle, the absorbent material having a non-circular cross section.

2. The fluid absorber of claim 1 wherein the absorbent material has an ellipsoidal cross section.

3. The fluid absorber of claim 1 wherein the absorbent material comprises a plurality of flat surfaces extending longitudinally along the absorbent material.

4. The fluid absorber of claim 1 wherein the absorbent material is tapered toward the distal extremity of the handle.

5. The fluid absorber of claim 1 wherein the absorbent material comprises cotton.

6. The fluid absorber of claim 1 wherein the absorbent material is of a density that absorbs semen upon contact.

7. The fluid absorber of claim 1 wherein the handle comprises rolled paper.

8. A fluid absorber comprising a rigid handle, an absorbent material attached to a distal extremity of the handle, and a housing enclosing the absorbent material, the handle extending through an opening in a proximal extremity of the housing, the absorbent material having a non-circular cross section.

9. The fluid absorber of claim 8 wherein a distal extremity of the housing is petaled.

10. The fluid absorber of claim 8 wherein the housing comprises paper.

11. The fluid absorber of claim 8 wherein the absorbent material has an ellipsoidal cross section.

12. The fluid absorber of claim 8 wherein the absorbent material comprises a plurality of flat surfaces extending longitudinally along the absorbent material.

13. The fluid absorber of claim 8 wherein the absorbent material is tapered toward the distal extremity of the handle.

14. The fluid absorber of claim 8 wherein the absorbent material comprises cotton.

15. The fluid absorber of claim 8 wherein the absorbent material is of a density that absorbs semen upon contact.

16. The fluid absorber of claim 8 wherein the handle comprises rolled paper.

17. The fluid absorber of claim 8 and further comprising a grip carried by a proximal extremity of the housing, the handle extending through an opening in the grip.

18. A method of removing fluid from a vagina after intercourse, the method comprising:
removing any protective wrapper from a fluid absorber of the kind having absorbent material carried by a handle;
grasping the handle;
inserting the absorbent material into the vagina;
leaving the absorbent material in place for an interval to time sufficient to absorb fluid in the vagina;
grasping the handle;
pulling the absorbent material out of the vagina; and
discarding the fluid absorber.

19. The method of claim 18 wherein the interval of time is about two minutes.

20. The method of claim 18 wherein inserting the absorbent material into the vagina comprises:
inserting the fluid absorber including a housing that encloses the absorbent material into the vagina, and
pressing the handle to eject the absorbent material from the housing and insert the absorbent material deeper into the vagina.
